(19) United States
(12) Patent Application Publication
(10) Pub. No.: US 2021/0289848 A1
(43) Pub. Date: Sep. 23, 2021

(54) PROTECTIVE PANTY/BIKINI WITH FLAVOR RIDGE FOR GUIDED LICKING

(71) Applicants: Melissa Mae Andrews, Reno, NV (US); Jeffrey Dean Parker, Othello, WA (US); Theresa Marie Hernandez, Sacramento, CA (US)

(72) Inventors: Melissa Mae Andrews, Reno, NV (US); Jeffrey Dean Parker, Othello, WA (US); Theresa Marie Hernandez, Sacramento, CA (US)

(21) Appl. No.: 17/338,756

(22) Filed: Jun. 4, 2021

Related U.S. Application Data
(63) Continuation-in-part of application No. 16/361,920, filed on Mar. 22, 2019.

(51) Int. Cl. A41B 9/00

(52) U.S. Cl. CPC A41B 9/004

(57) ABSTRACT

A protective panty/bikini has a shaped panel of moisture-resistant material that closely conforms to at least a portion of the crotch of a woman. The shaped panel has a waist opening, a right lea opening, a left leg opening, and a crotch portion that extends over the woman's crotch, closely covering the vulva and the anus. A flavor ridge runs centrally along the crotch portion, the flavor ridge extending over at least the vulva and the anus. The protective panty/bikini provides an erotic female garment that is convenient to wear, that accentuates womanly curves, and that provides a protective barrier against transmission of sexually transmitted disease during cunnilingus for both the wearer and her sexual partner. Additionally, the flavor ridge imparts a pleasant flavor, while providing lingual and tactile guidance to the woman's partner during cunnilingus in an environment with possibly limited visual guidance.

### CROSS-REFERENCE TO RELATED APPLICATIONS

The present application is a Continuation-in-Part of U.S. patent application Ser. No. 16/361,920, filed Mar. 22, 2019, titled “A PROTECTIVE FLAVORED THONG” which is hereby incorporated by reference in its entirety.

### FIELD OF THE INVENTION

This invention relates generally to female garments, and more particularly to prophylactic garments for enhancing oral erotic interactions.

### BACKGROUND OF THE INVENTION

As part of an erotic interaction, it is common for female participants to wear under-garments that can enhance a sexual experience. Common examples of female under-garments that provide increased sexual titillation are: bras, panties, bikinis, and G-strings.

A panty and a bikini bottom are both articles of female underwear that include a narrow strip of fabric or other material that passes between a woman's thighs and connects to a waistband. Women often wear panties or bikini bottoms to increase a sense of erotic excitement between themselves and their partners while engaging in sexual activities.

Panties and bikini bottoms can increase a woman's attractiveness in several ways. Because a panty or a bikini bottom is a skimpy and very revealing article of clothing, much of the woman's skin is exposed, enhancing her erotic appeal. In addition, panties and bikini bottoms are often tailored to accentuate the curves of a woman's body, and panties and bikini bottoms can include feminine decorations such as feminine colors, or lacey fabrics.

Panties and bikini bottoms are usually made from materials such as cotton, Rayon®, polyester, and/or Spandex®. Unfortunately, these materials will allow the passage of bodily fluids containing bacteria and viruses, and therefore typically panties and bikini bottoms cannot serve as a prophylactic undergarment for preventing the transmission of sexually transmitted diseases (STDs) such as syphilis and chlamydia (bacterial), and herpes, hepatitis C, and HPV (viral).

An example of a prophylactic undergarment is taught in Wallace, US Patent Application 2006/0042639 A1. Wallace teaches a prophylactic undergarment for use by a female wearer that includes an elastomeric portion that reduces the transmission of STDs. However, the woman's garment of Wallace has disadvantages. For example, the garment includes two prophylactic tubes configured to be inserted into the woman's vaginal canal and anus. Therefore the garment is difficult to put on, and may be uncomfortable to wear because of the two intrusive tubes.

### SUMMARY

The protective panty/bikini of the invention provides an erotic female under-garment that is convenient to wear, visually appealing, accentuates the wearer's natural womanly curves, and provides protection against transmission of STDs for both the wearer and her sexual partner during oral sex.

In addition, the protective panty/bikini of the invention includes a flavor ridge that runs along a central axis of the crotch that provides a pleasant scent and flavor, while also providing lingual and/or tactile guidance to the mouth and/or tongue of the wearer's partner while he/she performs cunnilingus in a dark environment.

The protective panty/bikini of the invention can include a panel of low-density polyethylene (LDPE) (similar to Saran Wrap®) that acts as a protective barrier during cunnilingus to protect against transmission of STDs between the wearer and her partner.

Another benefit of the protective flavored panty of the invention is that it is disposable.

A general aspect of the invention is a protective panty/bikini for snug wear on a lower trunk of a woman, the lower trunk having a right leg, a left leg, a waist, and a crotch, the crotch having a vulva and an anus. The protective panty/bikini includes: a shaped panel of moisture-resistant material configured to closely conform to at least a portion of the crotch, the shaped panel having: a waist opening configured to surround the waist, a right leg opening configured to surround the right leg, a left leg opening configured to surround the left leg, and a crotch portion configured to extend between the right leg opening and the left leg opening that is also configured to closely cover the vulva and the anus; and at least one flavor ridge applied centrally along the crotch portion so as to extend over at least the vulva and the anus.

In some embodiments, the protective panty/bikini further includes: a waistband configured to extend circumferentially around the waist opening; a right leg band configured to extend circumferentially around the right leg opening; and a left leg band configured to extend circumferentially around the left leg opening.

In some embodiments, the shaped panel is made from low-density polyethylene (LDPE).

In some embodiments, the shaped panel is made from latex.

In some embodiments, the shaped panel is made from a material that is impervious to fluids.

In some embodiments, the waistband includes a band of elastic silicone; the right leg band includes a band of elastic silicone; and the left leg band includes a band of elastic silicone.

In some embodiments, the at least one flavor ridge includes at least one of: a flavoring agent, a souring agent, and a sweetening agent.

In some embodiments, the protective panty/bikini further includes: a protective strip removably attached along the at least one flavor ridge, the protective strip being configured to protect freshness of the at least one flavor ridge while the at least one protective strip is attached to the at least one flavor ridge, and to be removable so as to leave the at least one flavor ridge substantially in place after the protective strip is removed.

In some embodiments, the at least one flavor ridge includes a pair of flavor ridges symmetrically placed along a central axis of the crotch portion.

In some embodiments, the protective panty/bikini further includes: a backing layer, the backing layer applied centrally along the crotch portion, the flavor ridge being applied centrally along the backing layer.

In some embodiments, the backing layer includes a coating of light adhesive that improves the adhesion of the backing layer to the crotch portion.

In some embodiments, the backing layer includes a smooth front-facing surface that retains its shape so as to facilitate licking of the flavor ridge off of the backing layer.

In some embodiments, the protective panty/bikini is packaged in a folded condition.

In some embodiments, the at least one flavor ridge folds once or twice when packaged in the folded condition.

In some embodiments, the at least one flavor ridge is flavored to resemble at least one of: lemon, lime, orange, cherry, pineapple, grape, blueberry, licorice, chocolate, cotton candy.

### BRIEF DESCRIPTION OF THE DRAWINGS

The invention will be more fully understood from the following Detailed Description, in conjunction with the following figures, wherein:

FIG. 1 is a front view of a protective panty embodiment having a flavor ridge, also showing a waistband extending around the waist opening of the protective panty; a right leg band extending around the right leg opening; and a left leg band extending around the left leg opening.

FIG. 2 is a front side view showing the flavor ridge on the protective panty embodiment of FIG. 1, also showing a protective strip being peeled off so as to expose the flavor ridge.

FIG. 3 is a rear side view of the protective panty embodiment of FIGS. 1 and 2, showing the flavor ridge extending from the front to the rear, and showing the protective strip being removed from the rear portion of the flavor ridge.

FIG. 4 is a front view of a protective bikini embodiment having a flavor ridge, also showing a waistband extending around the waist opening of the protective panty; a right leg band extending around the right leg opening; and a left leg band extending around the left leg opening.

FIG. 5 is a front side view showing the flavor ridge on the protective bikini embodiment of FIG. 4, also showing a protective strip being peeled off so as to expose the flavor ridge.

FIG. 6 is a rear side view of the protective bikini embodiment of FIGS. 4 and 5, showing the flavor ridge extending from the front to the rear, and showing the protective strip being removed from the rear portion of the flavor ridge.

FIG. 7 is a front view of a protective string bikini embodiment having a flavor ridge, also showing a waistband extending around the waist opening of the protective bikini; a right leg band extending around the right leg opening; and a left leg band extending around the left leg opening.

FIG. 8 is a front side view showing the flavor ridge on the protective string bikini embodiment of FIG. 7, also showing a protective strip being peeled off so as to expose the flavor ridge.

FIG. 9 is a rear side view of the protective string bikini embodiment of FIGS. 7 and 8, showing the flavor ridge extending from the front to the rear, and showing the protective strip being removed from the rear portion of the flavor ridge.

### DETAILED DESCRIPTION

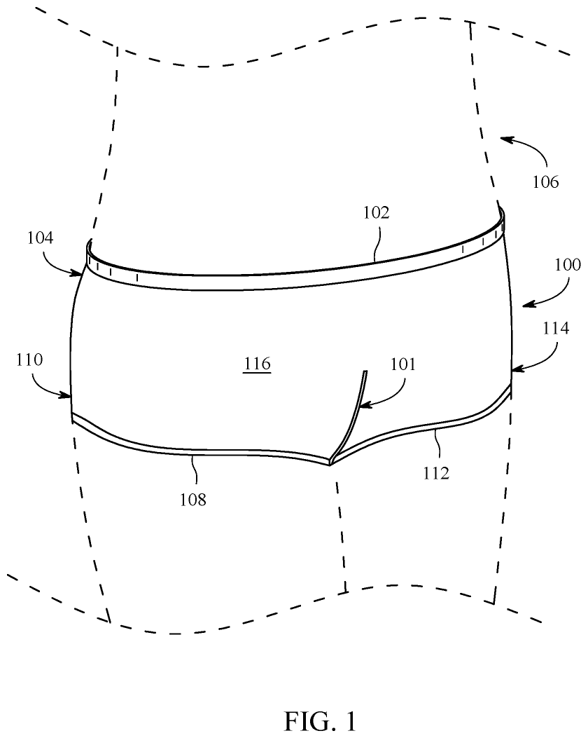

FIG. 1 is a front view of a protective panty embodiment 100 worn by a woman 106 with legs closed. The protective panty 100 has a flavor ridge 101, and a waistband 102 extending around the waist opening 104 of the protective panty 100. A right leg band 108 extends around the right leg opening 110. A left leg band 112 extends around the left leg opening 114. The waistband 102 includes a band of elastic silicone. Also, the right leg band 108 includes a band of elastic silicone, and the left leg band 112 includes a band of elastic silicone.

The protective panty 100 is worn snugly on the lower trunk of a woman 106, and is shaped so as to snugly cover the upper portion of her right and left legs, her lower waist, and her crotch, including snugly covering her vulva and anus.

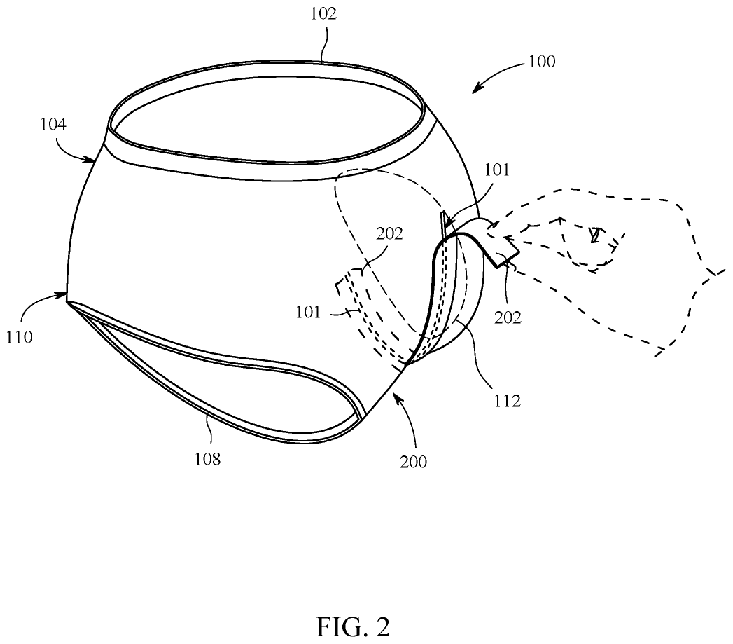

The protective panty 100 is made from a shaped panel 116 of moisture-resistant material configured to closely conform to the woman's crotch, both with legs open and leas closed. The shaped panel 116 includes the waist opening 104 configured to surround the waist, a right leg opening 110 configured to surround the right leg, a left leg opening 114 configured to surround the left leg, and a crotch portion 200 (see FIG. 2) configured to extend between the right leg opening 110 and the left leg opening 114. The crotch portion 200 also extends so as to closely cover the vulva and the anus of the woman 106. Also, at least one flavor ridge 101 extends centrally along the crotch portion 200 so as to extend over at least the vulva and the anus of the woman 106. The at least one flavor ridge 101 can be flavored to resemble at least one of: lemon, lime, orange, cherry, pineapple, grape, blueberry, licorice, chocolate, cotton candy. The at least one flavor ridge includes at least one of: a flavoring agent, a souring agent, and a sweetening agent. There can be one flavor ridge, or there can be two or three flavor ridges running in parallel centrally along the crotch portion 200 so as to extend over at least the vulva and the anus of the woman 106.

The shaped panel 116 can be made from low-density polyethylene (LDPE), or latex, or other material that is impervious to fluids.

FIG. 2 is a front side view of the protective panty 100 showing the flavor ridge 101 of the protective panty embodiment 100 of FIG. 1, also showing a protective strip 202 being peeled off so as to expose the flavor ridge 101. The protective strip 202 protects the flavor ridge 101 from air, dust, and bacteria. The protective strip 202 has a layer of adhesive that adheres to the flavor ridge 101 and to the crotch portion 200. The protective strip 202 is shown being manually removed just before use of the flavor ridge 101, thereby leaving the at least one flavor ridge 101 substantially in place after the protective strip is removed.

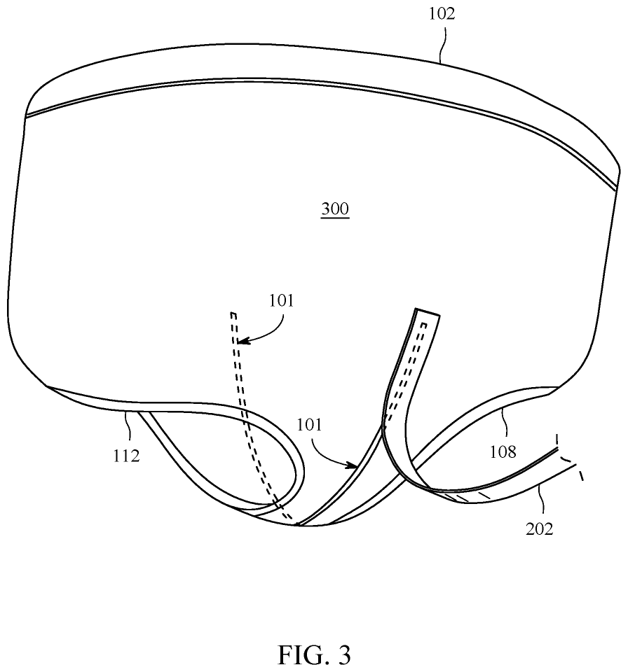

FIG. 3 is a rear side view of the protective panty embodiment 100 of FIGS. 1 and 2, showing the flavor ridge 101 extending from the front of the protective panty 100 to the rear 300, and showing the protective strip 202 being removed from the rear portion of the flavor ridge 101.

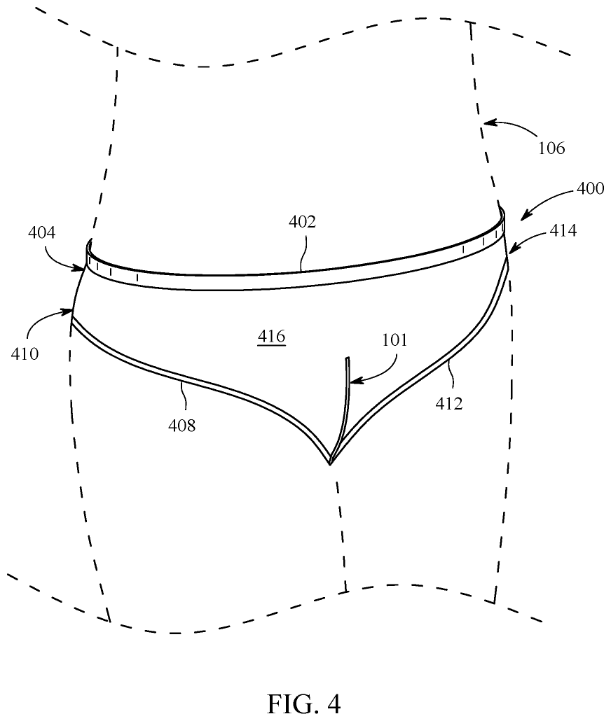

FIG. 4 is a front view of a protective bikini embodiment 400 worn by the woman 106 with legs closed. The protective bikini 400 has a flavor ridge 101, also showing a waistband 402 extending around the waist opening 404 of the protective bikini 400; a right leg band 408 extending around the right leg opening 410; and a left leg band 412 extending around the left leg opening 414.

The protective bikini 400 is worn snugly on the lower trunk of the woman 106 (configured to be worn lower on the lower trunk than the embodiment 100 of FIG. 1), and is shaped so as to snugly cover the upper portion of her right and left legs, her lower waist, and her crotch, including snugly covering her vulva and anus.

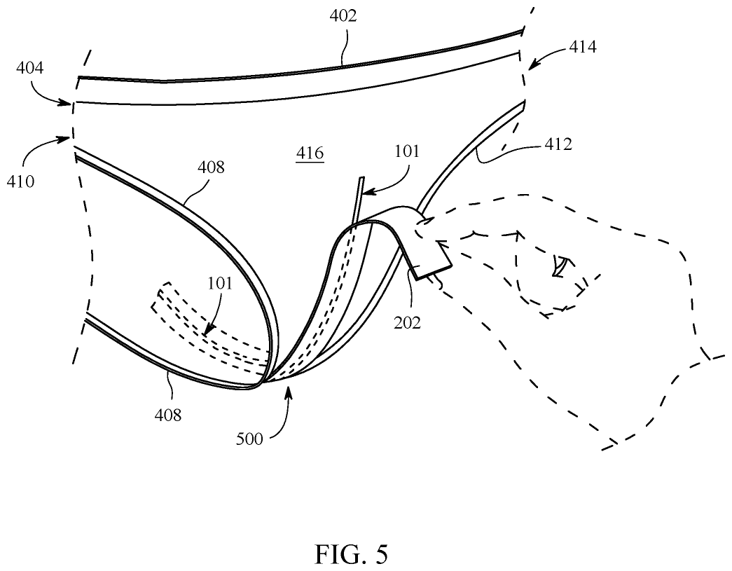

The protective bikini 400 is made from a shaped panel 416 of moisture-resistant material configured to closely conform to the woman's crotch, both with legs open and legs closed. The shaped panel includes the waist opening 404 configured to surround the waist, a right leg opening 410 configured to surround the right leg, a left leg opening 414 configured to surround the left leg, and a crotch portion 500 (see FIG. 5) configured to extend between the right leg opening 410 and the left leg opening 414. The crotch portion 500 also extends so as to closely cover the vulva and the anus of the woman 106. Also, at least one flavor ridge 101 extends centrally along the crotch portion 500 so as to extend over at least the vulva and the anus of the woman 106. In the protective bikini 400 embodiment, the waist opening 404 is configured to rise lower on the waist than the waist opening 104 of the panty embodiment 100, the right leg opening 410 is configured to rise higher on the right leg than the right leg opening 110 of the panty embodiment 100, and the left leg opening 414 is configured to rise higher on the left leg than the left leg opening 114 of the panty embodiment 100.

FIG. 5 is a front side view of the protective bikini 400 showing the flavor ridge 101 of the protective bikini embodiment 400 of FIG. 4, also showing the protective strip 202 being peeled off so as to expose the flavor ridge 101. The protective strip 202 protects the flavor ridge 101 from air, dust, and bacteria. The protective strip 202 has a layer of adhesive that adheres to the flavor ridge 101 and to the crotch portion 500. The protective strip 202 is shown being manually removed just before use of the flavor ridge 101.

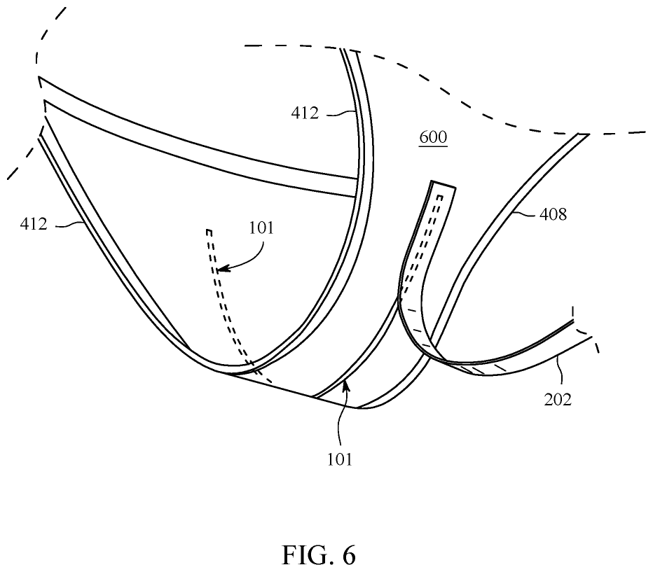

FIG. 6 is a rear side view of the protective bikini embodiment 400 of FIGS. 4 and 5, showing the flavor ridge 101 extending from the front of the protective bikini 400 to the rear 600, and showing the protective strip 202 being removed from the rear portion of the flavor ridge 101.

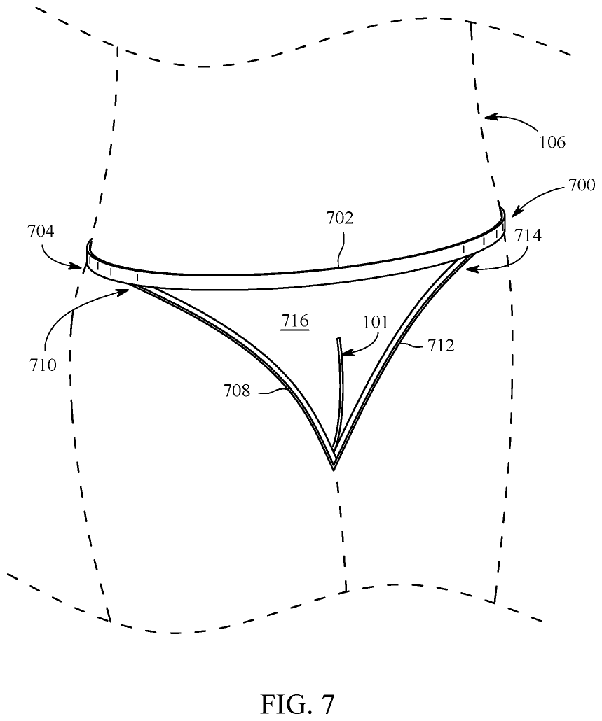

FIG. 7 is a front view of a protective string bikini embodiment 700 worn by the woman 106 with legs closed. The protective string bikini 700 has a flavor ridge 101, also showing a waistband 702 extending around the waist opening 704 of the protective string bikini 700; a right leg band 708 extending around the right leg opening 710; and a left leg band 712 extending around the left leg opening 714.

The protective string bikini 700 is worn snugly on the lower trunk of the woman 106 (configured to be worn lower on the lower trunk than the embodiment 400 of FIG. 4), and is shaped so as to snugly cover the upper portion of her right and left legs, her lower waist, and her crotch, including snugly covering her vulva and anus.

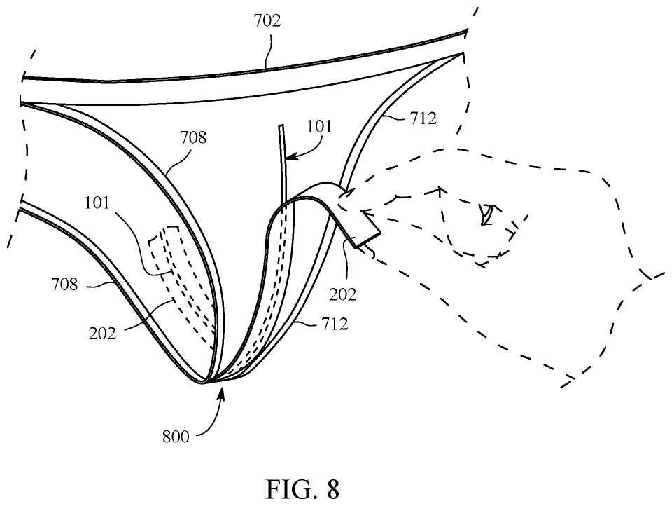

The protective string bikini 700 is made from a shaped panel 716 of moisture-resistant material configured to closely conform to the woman's crotch, both with legs open and legs closed. The shaped panel includes the waist opening 704 configured to surround the waist, a right leg opening 710 configured to surround the right leg, a left leg opening 714 configured to surround the left leg, and a crotch portion 800 (see FIG. 8) configured to extend between the right leg opening 710 and the left leg opening 714. The crotch portion 800 also extends so as to closely cover the vulva and the anus of the woman 106. Also, at least one flavor ridge 101 extends centrally along the crotch portion 800 so as to extend over at least the vulva and the anus of the woman 106. In the protective string bikini 700 embodiment, the waist opening 704 is configured to rise lower on the waist than the waist opening 404 of the bikini embodiment 400, the right leg opening 710 is configured to rise higher on the right leg than the right leg opening 410 of the bikini embodiment 400, and the left leg opening 714 is configured to rise higher on the left leg than the left leg opening 414 of the bikini embodiment 400.

FIG. 8 is a front side view of the protective bikini 700 showing the flavor ridge 101 of the protective bikini embodiment 700 of FIG. 7, also showing the protective strip 202 being peeled off so as to expose the flavor ridge 101. The protective strip 202 protects the flavor ridge 101 from air, dust, and bacteria. The protective strip 202 has a layer of adhesive that adheres to the flavor ridge 101 and to the crotch portion 800. The protective strip 202 is shown being manually removed just before use of the flavor ridge 101.

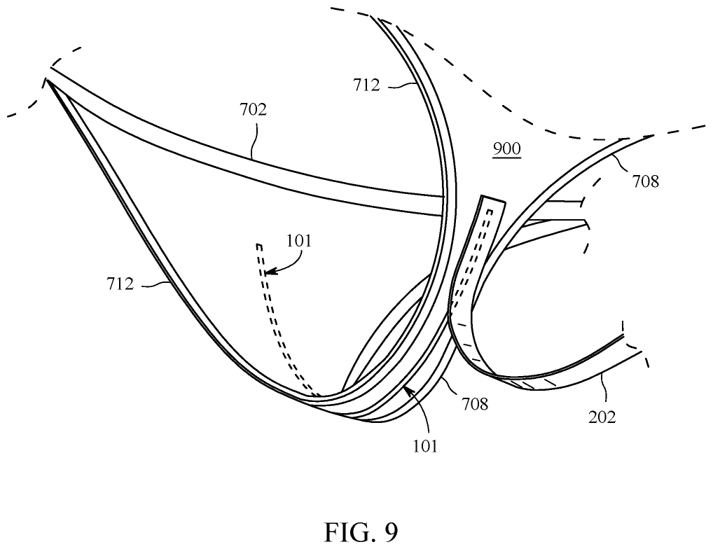

FIG. 9 is a rear side view of the protective bikini embodiment 700 of FIGS. 7 and 8, showing the flavor ridge 101 extending from the front of the protective string bikini 700 to the rear 900, and showing the protective strip 202 being removed from the rear portion of the flavor ridge 101.

In some embodiments, a backing layer can be applied centrally along the crotch portion, and then the flavor ridge can be applied centrally over the backing layer. The back surface of the backing layer can include a coating of light adhesive that improves the adhesion of the backing layer to the crotch portion. The flavor ridge 101 is applied to the front surface of the backing layer, the front surface being a smooth front-facing surface that retains its shape and supports the flavor ridge 101 so as to facilitate licking of the flavor ridge 101 off of the backing layer.

The protective panty/bikini 100, 400, 700 can be packaged in a folded condition. The at least one flavor ridge 101 folds once or twice when packaged in the folded condition.

Other modifications and implementations will occur to those skilled in the art without departing from the spirit and the scope of the invention as claimed. Accordingly, the above description is not intended to limit the invention, except as indicated in the following claims.

What is claimed is:

1. A protective panty/bikini for snug wear on a lower trunk of a woman, the lower trunk having a right leg, a left leg, a waist, and a crotch, the crotch having a vulva and an anus, the protective panty/bikini comprising:
a shaped panel of moisture-resistant material configured to closely conform to at least a portion of the crotch, the shaped panel having:
a waist opening configured to surround the waist,
a right leg opening configured to surround the right leg,
a left leg opening configured to surround the left leg, and
a crotch portion configured to extend between the right leg opening and the left leg opening that is also configured to closely cover the vulva and the anus; and
at least one flavor ridge applied centrally along the crotch portion so as to extend over at least the vulva and the anus.

2. The protective panty/bikini of claim 1, further comprising:
a waistband configured to extend circumferentially around the waist opening;
a right leg band configured to extend circumferentially around the right leg opening; and
a left leg band configured to extend circumferentially around the left leg opening.

3. The protective panty/bikini of claim 1, wherein the shaped panel is made from low-density polyethylene (LDPE).

4. The protective panty/bikini of claim 1, wherein the shaped panel is made from latex.

5. The protective panty/bikini of claim 1, wherein the shaped panel is made from a material that is impervious to fluids.

6. The protective panty/bikini of claim 1, wherein
the waistband includes a band of elastic silicone;
the right leg band includes a band of elastic silicone; and
the left leg band includes a band of elastic silicone.

7. The protective panty/bikini of claim 1, wherein the at least one flavor ridge includes at least one of: a flavoring agent, a souring agent, and a sweetening agent.

8. The protective panty/bikini of claim 1, further comprising:
a protective strip removably attached along the at least one flavor ridge, the protective strip being configured to protect freshness of the at least one flavor ridge while the at least one protective strip is attached to the at least one flavor ridge, and to be removable so as to leave the at least one flavor ridge substantially in place after the protective strip is removed.

9. The protective panty/bikini of claim 1, wherein the at least one flavor ridge includes a pair of flavor ridges symmetrically placed along a central axis of the crotch portion.

10. The protective panty/bikini of claim 1, further comprising:
a backing layer, the backing layer applied centrally along the crotch portion,
the flavor ridge being applied centrally along the backing layer.

11. The protective panty/bikini of claim 10, wherein the backing layer includes a coating of light adhesive that improves the adhesion of the backing layer to the crotch portion.

12. The protective panty/bikini of claim 10, wherein the backing layer includes a smooth front-facing surface that retains its shape so as to facilitate licking of the flavor ridge off of the backing layer.

13. The protective panty/bikini of claim 1, wherein the protective panty/bikini is packaged in a folded condition.

14. The protective panty/bikini of claim 13, wherein the at least one flavor ridge folds once or twice when packaged in the folded condition.

15. The protective panty/bikini of claim 1, wherein the at least one flavor ridge is flavored to resemble at least one of: lemon, lime, orange, cherry, pineapple, grape, blueberry, licorice, chocolate, cotton candy.
